(19) United States
(12) Patent Application Publication
(10) Pub. No.: US 2025/0026642 A1
(43) Pub. Date: Jan. 23, 2025

(54) SILICON CARBIDE DEVICES AND METHODS OF MAKING AND USING THE SAME

(71) Applicant: The University of North Carolina at Charlotte, Charlotte, NC (US)

(72) Inventor: Ahmed EI-Ghannam, Charlotte, NC (US)

(21) Appl. No.: 18/776,719

(22) Filed: Jul. 18, 2024

Related U.S. Application Data
(60) Provisional application No. 63/527,404, filed on Jul. 18, 2023.

(51) Int. Cl. C01B 21/068; C04B 35/565

(52) U.S. Cl. CPC C01B 21/0682; C04B 35/565

(57) ABSTRACT

The present invention relates to silicon carbide (SiC) devices having a surface that has been activated to enhance properties including strength, porosity, and bioactivity. Activation may include forming silica gel with or without silicon nitride on the surface. The invention further relates to methods of making the devices and using the devices.

### RELATED APPLICATION INFORMATION

This application claims the benefit of U.S. Provisional Application Ser. No. 63/527,404, filed Jul. 18, 2023, the disclosure of which is incorporated herein by reference in its entirety.

### FIELD OF THE INVENTION

The present invention relates silicon carbide (SiC) devices having a surface that has been activated to enhance properties including strength, porosity, and bioactivity. The invention further relates to methods of making and using the devices.

### BACKGROUND OF THE INVENTION

Ceramic additive manufacturing is a process that builds ceramic parts layer by layer, directly from digital models. It is commonly known that this technique offers a stark contrast to traditional manufacturing methods, which involve subtractive processes, such as cutting or drilling, which can waste material and limit design possibilities. At present, ceramic additive manufacturing methods require the use of binders to bond the ceramic particle during layer-by-layer 3D printing of components. The use of polymer or low melting glass binder has many limitations including non-homogeneity of the ceramic-polymer suspension, limited ceramic load in the polymer medium, component deformation during printing, the risk of collapse during de-binding and high porosity of the final product. Additionally, the use of high energy lasers to sinter ceramic particles is challenged by the high melting point of ceramics and the thermal stresses created during printing. These processes can lead to defects and distortions in the structure, affecting the reliability of the final product.

Regarding medical applications, any changes in the composition and structure of the 3D printed ceramic implants may affect its clinical performance. Consequently, research in ceramic additive manufacturing is still in its foundational stages, facing various unresolved challenges compared to other materials. Extensive research activities have primarily focused on controlling particle size and surface properties of ceramic materials. Additionally, interactions between binders and dispersants to enhance the overall quality of ceramic additive manufacturing products are being scrutinized.

Silicon carbide (SiC) offers desirable material properties in ceramic additive manufacturing, but the difficulty in its manufacture currently limits the application opportunities. For example, in space-based optics fabrication applications, SiC appears to be an ideal optical material from a material properties standpoint, yet the cost and lead-time associated with the preliminary shaping, light weighting, and optical finishing have been major impediments in its use of the material in optical systems.

Furthermore, there are currently many limitations with the SiC manufacturing process. For example, when the Hot-pressed method is used, micrometer-scale alpha phase SiC particles are consolidated in a mold at high temperatures (>2000° C.) and pressures (1000-2000 atm) to form simple shapes, and thus, the material removal rates are low in SiC (<5% of the rates for glasses and metals due to its high hardness and strength), where the secondary processing steps are time-consuming and costly.

Another example is when the Chemical Vapor Deposition (CVD) method is used. An organo-metallic vapor is deposited onto a heated substrate (>1300° C.). This gives a very limiting beta (cubic) phase SiC coating with fine columnar grains and is typically used as a layer on top of an existing structure (e.g., graphite or SiC) to encapsulate the surface. For the Reaction Bonded method, a SiC alpha (hexagonal) phase slurry is slip cast (injected) into a mold with the negative of the desired geometry. This part is then removed from the mold and, if required, the part can be machined to modify the geometry. The part is then heated to sinter the SiC particles, which has an open porosity and filled with free silicon in a second heating operation (i.e., reaction bonding). The final microstructure offers a uniform dispersion of Si and SiC regions and is typically finish machined to produce the desired part. Reaction bonding produces near-net shapes and therefore offers reduced machining time, but at the cost of a mold which can be expensive for low-volume production.

In an additional example, when Graphite Conversion is used, POCO Graphite offers a graphite preform, SUPERSiC, which can be machined to a near-net shape. The graphite is then purified and subjected to a proprietary conversion process which substitutes silicon atoms for carbon atoms to obtain SiC. This conversion process is limited to a 3 mm depth (6 mm total thickness) and the final part usually requires an additional step of a CVD coat to improve the optical finishing properties.

In addition to these “traditional” manufacturing processes described above, additive manufacturing was recently tested for alumina (or aluminum oxide, Al₂O₃) parts. In this study, the performance of sintered alumina was compared to 3D printed alumina specimens for ballistic protection, where the ultimate application is customized armor for soldiers, vehicles, and equipment. Pressurized spray deposition of a proprietary blend of ceramic powder and a polymeric binder was applied layer-by-layer to produce the 3D printed samples. After printing, sintering was completed to remove the polymer; this naturally caused significant shrinkage due to the polymer loss and added the requirement for post-process grinding to achieve tight dimensional tolerances. However, testing demonstrated that the sintered alumina was only 13% more effective against ballistic penetration than the 3D printed alumina specimens.

Therefore, while many additive manufacturing methods for SiC show improvement, widespread adoption has been hampered due to costs and limitations on shape. Thus, there is still a need for cost-effective methods and non-limiting structures.

### SUMMARY OF THE INVENTION

The present invention is based on the development of methods to chemically activate SiC ceramic surfaces through alkali treatments to form a silica gel layer and nitrogen treatments to form silicon nitride. The method creates a SiC device with enhanced physical properties, including strength, porosity, and bioactivity. The methods of the invention enable enhanced manufacturing processes without the use of polymer binders.

Thus, one aspect of the present invention relates to a method of making a SiC device, comprising: (i) preparing a SiC device; (ii) thermally treating the SiC device at about 500-900° C.; (iii) exposing the SiC device to NaOH to form a silica gel layer on the surface of the SiC device; (iv) thermally treating the SiC device after exposing at about 550-1300° C.; (v) exposing the SiC device to nitrogen to form silicon nitride on the SiC device surface; and (vi) thermally treating the SiC device after exposing to nitrogen.

In another aspect, the present invention relates to a method of increasing strength of a SiC device, comprising: (i) preparing a SiC device; (ii) thermally treating the SiC device at about 500-900° C.; (iii) exposing the SiC device to NaOH to form a silica gel layer on the surface of the SiC device; (iv) thermally treating the SiC device after exposing at about 550-1300° C.; (v) exposing the SiC device to nitrogen to form silicon nitride on the SiC device surface; and (vi) thermally treating the SiC device after exposing to nitrogen.

In an additional aspect, the present invention relates to a method of increasing bioactivity of a SiC device, comprising: (i) preparing a SiC device; (ii) thermally treating the SiC device at about 500-900° C.; (iii) exposing the SiC device to NaOH to form a silica gel layer on the surface of the SiC device; (iv) thermally treating the SiC device after exposing at about 550-1300° C.; (v) exposing the SiC device to nitrogen to form silicon nitride on the SiC device surface; and (vi) thermally treating the SiC device after exposing to nitrogen.

In another aspect, the present invention relates to a method of increasing biocompatibility of a SiC device, comprising: (i) preparing a SiC device; (ii) thermally treating the SiC device at about 500-900° C.; (iii) exposing the SiC device to NaOH to form a silica gel layer on the surface of the SiC device; (iv) thermally treating the SiC device after exposing at about 550-1300° C.; (v) exposing the SiC device to nitrogen to form silicon nitride on the SiC device surface; and (vi) thermally treating the SiC device after exposing to nitrogen.

In an additional aspect, the present invention relates to a method of increasing porosity of a SiC device, comprising: (i) preparing a SiC device; (ii) thermally treating the SiC device at about 500-900° C.; (iii) exposing the SiC device to NaOH to form a silica gel layer on the surface of the SiC device; (iv) thermally treating the SiC device after exposing at about 550-1300° C.; (v) exposing the SiC device to nitrogen to form silicon nitride on the SiC device surface; and (vi) thermally treating the SiC device after exposing to nitrogen.

In a further aspect, the present invention relates to a method of increasing strength of a SiC device, comprising: (i) exposing SiC particles to NaOH to create a silica gel layer; (ii) drying the SiC particles in air to dry the silica-gel layer; (iii) mixing the SiC particles with aluminium oxide and/or magnesium oxide and/or silicate mineral particles such as mullite, cordierite, cristobalite, or spodumene; (iv) preparing a SiC device using the mixture of step (iii), e.g., using a 3D printer or by pressing in a mold; and (v) thermally treating the SiC device at about 800-1400° C. for 4-24 hours.

In another aspect, the present invention relates to SiC devices produced using the methods of the invention, e.g., wherein the SiC device is a medical device, ground-based imaging system device or a space-based imaging system device, a lithography positioning tool or a stepper system device, a scanning mirror system device or optoelectronic device, a ballistic protection device, an energy or nuclear power device, a component of an automotive device, a component in an electrical vehicle, or a component of a filter device.

In an additional aspect, the present invention relates to a method of implanting or attaching a SiC device to a subject in need thereof, comprising using a SiC device prepared by the methods of the invention.

In a further aspect, the present invention relates to a method of stimulating innervation of tissue in a subject in need thereof, comprising contacting the tissue with a SiC device prepared by the methods of the invention.

These and other aspects of the invention are set forth in more detail in the description of the invention below.

### BRIEF DESCRIPTION OF THE DRAWINGS

The accompanying drawings, which are incorporated in and constitute a part of the specification, illustrate embodiments of the invention and, together with the description, serve to explain principles of the invention.

FIGS. 1A-1B show (FIG. 1A) 3D printed SiC screw digital image and (FIG. 1B) SEM image of screw threads after protocol #2 post-processing treatment.

FIGS. 2A-2C shows a stress-strain curve obtained from compression test performed on samples prepared by following post-processing protocol #1 (FIG. 2A) or #2 (FIG. 2B). Representative digital image of fractured sample prepared by using post-processing protocol #2 (FIG. 2C).

FIGS. 3A-3B show a scanning electron microscopy (SEM) analysis of (FIG. 3A) outer surface, and (FIG. 3B) inner surface of the sample prepared according to protocol #2.

FIG. 4A shows deconvolution of FTIR spectra of 3D printed samples subjected to protocol #1.

FIG. 4B shows deconvolution of FTIR spectra of 3D printed samples subjected to protocol #2 post-processing treatment. 3D printed SiC samples treated according to protocol #2 post-processing depicted bands characteristic of Si—N bond.

FIG. 5A shows X-ray diffraction (XRD) analysis showing the characteristic signals for α-SiC and β-cristobalite (SiO₂) solid solution phases for the sample treated using protocol #1.

FIG. 5B shows XRD analysis showing the characteristic signals for α-SiC, Si₃N₄ and tridymite (SiO₂) solid solution phases of the sample prepared by following protocol #2.

FIG. 6A shows SEM analysis of fracture surface of SiC screw samples following a 550° C./20 min treatment.

FIG. 6B shows an energy dispersive X-Ray (EDX) compositional analysis of the silica layer.

FIG. 7 shows the FTIR deconvolution of the 3D printed SiC orthopedic screw treated according to protocol #1 and immersed in a simulated body fluid (SBF) for 3, 6, and 11 days. Evidence of precipitation of hydroxyapatite layer after 3 days of immersion in SBF is supported by the presence of bands at 590 and 619 cm⁻¹.

FIGS. 8A-8B show SEM analysis of the sample surface at ×4,000 (FIG. 8A) and ×10,000 (FIG. 8B) magnification showing the hydroxyapatite layer deposited onto the surface of 3D printed SiC screws following immersion in SBF for 11 days.

FIGS. 9A-9B shows the inductively Coupled Plasma-optical Emission Spectrometry (ICP-EOS) ionic concentrations of Si and titanium in tissue culture media diluted with 10, 25 and 50% PBS preincubated with 3D printed SiC screws (FIG. 9A) and titanium alloy discs (FIG. 9B). N=3; ANOVA and post-hoc tests with *p<0.05; **p<0.01; ***p<0.001; ****p<0.0001.

FIG. 10A shows the effects of Si and titanium dissolution products on J774 macrophage stress, i.e., reactive oxygen species (ROS) expression. Following a 24-hr incubation with increasing concentrations of SiC and Ti alloy dissolution products (10:90; 25:75; 50:50 dilutions, respectively), J774 macrophage ROS expression and cell death were determined. ANOVA and post-hoc tests were used to determine concentrations effects with *p<0.05, **p<0.01, ***p<0.001 and p<0.0001.

FIG. 10B shows the effects of Si and titanium dissolution products on J774 macrophage stress, i.e., cell membrane degradation. Following a 24-hr incubation with increasing concentrations of SiC and Ti alloy dissolution products (10:90; 25:75; 50:50 dilutions, respectively), J774 macrophage ROS expression and cell death were determined. ANOVA and post-hoc tests were used to determine concentrations effects with *p<0.05, **p<0.01, ***p<0.001 and p<0.0001.

FIG. 11A shows the effects of Si and titanium dissolution products on J774 macrophage TNFα secretions. Following a 24-hr incubation with increasing concentrations of SiC and titanium material dissolution products (10:90; 25:75; 50:50 dilutions, respectively), TNFα and IL6R present in J774 macrophage media were determined by ELISA. ANOVA and post-hoc tests were used to determine concentrations effects with *p<0.05, **p<0.01, ***p<0.001 and p<0.0001.

FIG. 11B shows the effects of Si and titanium dissolution products on J774 macrophage IL6R shedding. Following a 24-hr incubation with increasing concentrations of SiC and titanium material dissolution products (10:90; 25:75; 50:50 dilutions, respectively), TNFα and IL6R present in J774 macrophage media were determined by ELISA. ANOVA and post-hoc tests were used to determine concentrations effects with *p<0.05, **p<0.01, ***p<0.001 and p<0.0001.

FIGS. 12A-12D show bone cells attachment and spreading on the surface of 3D printed SiC screw. After 6 days in culture, the surface of the 3D printed screws was covered by layers of extracellular matrix produced by bone cells (FIG. 12A). Higher magnifications show individual cells attached and spreading onto the SiC surface (FIG. 12B). Moreover, bundles of fused collagenous matrix (FIGS. 12C and 12D) filled and bridged the screw pores.

FIG. 13A shows the primary neurons, dorsal root ganglion (DRG), grown onto SiC disc and were cultured and their growth onto SiC discs demonstrated by active metabolism (Calcein AM staining). Representative microphotographs obtained using phase contrast and fluorescence microscopy (scale bar=100 μm).

FIG. 13B shows the presence of axons networks is shown in SEM microphotographs of DRG neurons onto SiC discs.

FIGS. 14A-14B show (FIG. 14A) an immunolabeling with S100 antibody showing positive red staining (arrows) indicative of the innervation of the newly formed bone. A small nerve bundle (white arrow) can be seen attached to newly generated blood vessels within de novo bone. (FIG. 14B) shows the circle, which emphasizes the presence of multiple osteoblasts exhibiting positive bone sialoprotein expression. The arrows denote bone formation occurring within the pores of the SiC implant.

FIG. 15 shows a scheme of the chemical oxidation of SiC surface by NaOH solution. The NaOH breaks Si—C bonds and leading to the formation of silanol (Si—OH) groups and silanol groups polymerize to form a microscale silica layer.

FIG. 16 shows a schematic of the rehydration of the dried silica gel layer on SiC particles during printing when using an aqueous binder.

FIG. 17 shows the SEM fractured surface analysis of SiC disc treated at 900° C./2 h in air. Extensive growth of dense crystalline silica nano wires can be seen bridging the walls of the pores and connecting the SiC particles. The schematic on the right shows the linking of the SiO₄ tetrahedral to form chain molecules leading to nanowire growth.

FIG. 18 shows the mechanical properties of SiC-mineral composites.

FIG. 19 shows the electrical properties of SiC-mineral composites.

FIG. 20 shows the compressive strength of SiC-mineral composites.

### DETAILED DESCRIPTION OF THE INVENTION

The present invention now will be described hereinafter with reference to the accompanying drawings and examples, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items.

The transitional phrase “consisting essentially of” means that the scope of a claim is to be interpreted to encompass the specified materials or steps recited in the claim and those that do not materially affect the basic and novel characteristic(s) of the claimed invention.

It will be understood that, although the terms “first,” “second,” etc. may be used herein to describe various elements, and these elements should not be limited by these terms. These terms are only used to distinguish one element from another. Thus, a “first” element discussed below could also be termed a “second” element without departing from the teachings of the present invention. The sequence of operations (or steps) is not limited to the order presented in the claims or figures unless specifically indicated otherwise.

Furthermore, the term “about,” as used herein when referring to a measurable value such as an amount of a compound or agent of this invention, dose, time, temperature, and the like, is meant to encompass variations of ±10%, ±5%, ±1%, ±0.5%, or even ±0.1% of the specified amount.

The terms “silicon carbide” and “SiC” as used herein, refer to a hard chemical compound containing silicon and carbon that exists in about 250 crystalline forms. Silicon carbide can be manufactured by combining silica sand and carbon at a high temperature, e.g., in an Acheson graphite electric resistance furnace, between 1,600° C. (2,910° F.) and 2,500° C. (4,530° F.). Fine SiO₂ particles in plant material (e.g., rice husks) can be converted to SiC by heating in the excess carbon from the organic material. The silica fume, which is a by product of producing silicon metal and ferrosilicon alloys, can also be converted to SiC by heating with graphite at 1,500° C. (2,730° F.).

The term “preparing” as used herein with respect to SiC, refers to the art-recognized steps used to prepare a SiC device. The device may be prepared using any additive manufacturing methods. Preparing includes, without limitation, 3D printing, powder metallurgy methods, and Chemical Vapor Deposition (CVD).

The term “thermally treating” as used herein, refers to the art-recognized steps of heat treating (or heat treatment) where a group of industrial, thermal and metalworking processes are used to alter the physical, and sometimes chemical, properties of a material. The most common application is metallurgical. Heat treatment involves the use of heating the material to slightly elevated or extreme temperatures to achieve the desired result such as hardening of a material. Heat treatment techniques include, without limitation, annealing, case hardening, precipitation strengthening, tempering, carburizing, normalizing, and quenching.

The term “exposing” as used herein with respect to SiC, refers to the art-recognized steps of coming into contact with another substance through either direct or indirect contact. When the substance is a liquid, exposing may include submersion of the device or spraying the device with the substance. When the substance is a gas, exposing may include placing the device in an atmosphere of the substance.

The term “coating” as used herein with respect to SiC, refers to surface engineering where a layer (or multiple layers) of material is deposited onto a device to alter the surface properties.

“3D printing” as used herein, refers to 3D printing or additive manufacturing, which is a process of making three dimensional objects from a digital file. The creation of a 3D printed object is achieved using additive processes. In an additive process an object is created by laying down successive layers of material until the object is created. Each of these layers can be seen as a thinly sliced cross-section of the object. Volumetric 3D printing consists of printing entire structures that can be formed at once without the need for layer-by-layer fabrication. 3D printing is the opposite of subtractive manufacturing which is cutting out/hollowing out a block of material with, for instance, a milling machine.

The present invention is based on the development of methods to chemically activate SiC ceramic surfaces through alkali treatments to form a silica gel layer and nitrogen treatments to form silicon nitride. The method creates a SiC device with enhanced physical properties, including strength, porosity, and bioactivity. The methods of the invention enable enhanced manufacturing processes without the use of polymer binders.

Thus, one aspect of the present invention relates to a method of making a SiC device, comprising: (i) preparing a SiC device; (ii) thermally treating the SiC device at about 500-900° C.; (iii) exposing the SiC device to NaOH to form a silica gel layer on the surface of the SiC device; (iv) thermally treating the SiC device after exposing at about 550-1300° C.; (v) exposing the SiC device to nitrogen to form silicon nitride on the SiC device surface; and (vi) thermally treating the SiC device after exposing to nitrogen.

In another aspect, the present invention relates to a method of increasing strength of a SiC device, comprising: (i) preparing a SiC device; (ii) thermally treating the SiC device at about 500-900° C.; (iii) exposing the SiC device to NaOH to form a silica gel layer on the surface of the SiC device; (iv) thermally treating the SiC device after exposing at about 550-1300° C.; (v) exposing the SiC device to nitrogen to form silicon nitride on the SiC device surface; and (vi) thermally treating the SiC device after exposing to nitrogen.

In an additional aspect, the present invention relates to a method of increasing bioactivity of a SiC device, comprising: (i) preparing a SiC device; (ii) thermally treating the SiC device at about 500-900° C.; (iii) exposing the SiC device to NaOH to form a silica gel layer on the surface of the SiC device; (iv) thermally treating the SiC device after exposing at about 550-1300° C.; (v) exposing the SiC device to nitrogen to form silicon nitride on the SiC device surface; and (vi) thermally treating the SiC device after exposing to nitrogen.

In another aspect, the present invention relates to a method of increasing biocompatibility of a SiC device, comprising: (i) preparing a SiC device; (ii) thermally treating the SiC device at about 500-900° C.; (iii) exposing the SiC device to NaOH to form a silica gel layer on the surface of the SiC device; (iv) thermally treating the SiC device after exposing at about 550-1300° C.; (v) exposing the SiC device to nitrogen to form silicon nitride on the SiC device surface; and (vi) thermally treating the SiC device after exposing to nitrogen.

In an additional aspect, the present invention relates to a method increasing porosity of a SiC device, comprising: (i) preparing a SiC device; (ii) thermally treating the SiC device at about 500-900° C.; (iii) exposing the SiC device to NaOH to form a silica gel layer on the surface of the SiC device; (iv) thermally treating the SiC device after exposing at about 550-1300° C.; (v) exposing the SiC device to nitrogen to form silicon nitride on the SiC device surface; and (vi) thermally treating the SiC device after exposing to nitrogen.

In some embodiments, the SiC device is prepared by additive manufacturing, powder metallurgy, or CVD. Additive manufacturing is the process of creating an object by building it one layer at a time. To create an object using additive manufacturing, first a design is created by using a computer aided design, or CAD, software, or by taking a scan of the object that is to be printed. Software then translates the design into a layer-by-layer framework for the additive manufacturing machine to follow. This is sent to the 3-D printer, which begins creating the object immediately. Powder metallurgy is a metal-forming process performed by heating compacted metal powders to just below their melting points. It comprises several different technologies for fabricating semi-dense and fully dense components. CVD is a process in which gaseous or vapor substance reacts on a gas-solid interface to produce a deposition on the surface of the solid material.

In another embodiment, the SiC device is prepared by 3D printing the SiC device. 3D printing is a specific subset of additive manufacturing, and it refers to the process of creating three-dimensional objects from a digital model by depositing material layer by layer, usually using an extrusion-based method.

In an additional embodiment, the 3D printing comprises using SiC particles, wherein the 3D printing is performed using SiC particles having a size of about 10 nm to about 80 microns. To facilitate flow-ability and homogeneous layer dragging during printing, multi-modal particle size can be used. The multimodal materials make greater use of the 3D printing output from gas atomization while bringing productivity advantages to laser powder bed fusion and, increasingly, binder jetting. Multimodal materials lend themselves to denser packing of the material in the powder bed, leading to workflow advantages as well as improved mechanical strength in the final parts. Plus, working with a mix or blend of particle sizes provides a use for cuts that might otherwise be scrapped. In one embodiment, to facilitate flow-ability and homogeneous layer dragging during printing, multi-modal particle sizes may be used such as 40 micron, 2 micron and 0.6 micron. The selection of the particle sizes and the corresponding ratios were based on the size of the pores observed by SEM on the SiC discs. SiC is a hard, high-temperature-resistant material with excellent mechanical-thermal properties. While traditional manufacturing processes involve complex and multi-step methods and processing, strongly limiting design freedom, 3D printing of SiC offers unique advantages such as enhanced complex geometries, customization, and reduction of material waste.

In an additional embodiment, the SiC particles used for 3D printing are pre-treated with NaOH. Treating SiC particles with NaOH creates a silica gel layer that enhances the bonding during thermal oxidation. Some or all of the SiC particles can be pre-treated with NaOH. In another embodiment, the pre-treatment of SiC particles with NaOH is carried out with about 15-20% NaOH. In one embodiment, the pre-treatment of SiC particles with NaOH is carried out with about 20% NaOH. In another embodiment, at least 20% of the surface of the SiC particles is coated in a silica gel layer created by the treatment with NaOH, e.g., about 20%-100%, e.g., at least 20%, 30%, 40%, 50%, 60%, 70%, 80%, or 90% or any range or value therein. The silica gel layer in a wet or dry format serves as a binder for the SiC particles at room temperature without a need for compact pressure. Therefore, SiC particles coated with dried silica gel layer can be used in a powder bed 3D printer to make devices using water as a binder. The spraying of water by the printer head will hydrate the dried silica gel layer coating the SiC particles. The hydrated silica gel enables bonding of the SiC particles through hydrogen bonds and Si—O—Si bonds. In another embodiment, the SiC particles are washed, dried, separated, and sifted prior to 3D printing.

In an additional embodiment, the 3D printing is performed at room temperature. 3D printing may be performed at a temperature between 15° C. and 30° C. to avoid problems that affect print quality. In very hot environments, material flow or shrinkage can be affected, leaving threads or bubbles on the piece surface. A very cold environment can cause various problems such as warping, which is the total or partial lifting of the corners of the model in contact with the printing surface due to the contraction of the material, usually caused by a very sudden cooling during the printing process; and delamination or cracking, which occurs due to the stresses induced by the suddenness of cold on the material.

In another embodiment, after the SiC device is prepared, the SiC device is thermally treated at about 500-900° C., e.g., about 500, 550, 600, 650, 700, 750, 800, 850, or 900° C. or any range therein, e.g., about 550-800° C., about 600-700° C., or about 650° C. In an additional embodiment, the thermal treatment of the SiC device is carried out for about 2-8 hours, e.g., about 2, 3, 4, 5, 6, 7, or 8 hours, e.g., about 4-6 hours. In one embodiment, the thermal treatment of the SiC device is carried out for about 5 hours.

In an additional embodiment, the thermal treatment is carried out in a carbon atmosphere, e.g., in the presence of graphite or carbon powder or a hydrocarbon such as methane or any organic material, e.g., in a carbon monoxide or argon atmosphere. The thermal treatment in a carbon atmosphere converts the silica bonding zone between SiC particles into a SiC zone. Specifically, the bonding zone between SiC particles in the 3D printed device acts like a sponge in the sense that it can accept the diffusion of atoms and ions in it. Heating the 3D printed SiC device in a carbon atmosphere will soften the silica in the bonding zone between SiC particles and this will permit the diffusion of ions or atoms into the silica bonding zone. The diffusion is motivated by the concentration gradient. The silica bonding zone is made of silicon and oxygen (silica). When heated in carbon atmosphere the carbon atoms tend to diffuse into the silica converting it from silicon oxide to silicon carbide. The conversion of the silica bonding zone to silicon carbide makes the entire structure of the device made of SiC, and the homogenous SiC structure acquires all the unique properties of pure SiC. In an additional embodiment, exposing the SiC device to NaOH forms a silica gel layer on the surface of the SiC device. In another embodiment, exposing the SiC device to NaOH is carried out with about 15-20% NaOH. In one embodiment, exposing the SiC device to NaOH is carried out with about 20% NaOH. In another embodiment, at least 20% of the surface of the SiC device is coated in a silica gel layer created by the treatment with NaOH, e.g., about 20%-100%, e.g., at least 20%, 30%, 40%, 50%, 60%, 70%, 80%, or 90% or any range or value therein. This silica gel layer serves as a precursor for growth of silica nanowires that bridge the walls between particles that subsequently densifies and strengthens the resulting device.

In another embodiment, after exposing the SiC device to NaOH to form a silica gel layer on the surface of the SiC device the SiC device is thermally treated at about 550-1300° C., e.g., about 550, 600, 650, 700, 750, 800, 850, 900, 1000, 1050, 1100, 1150, 1200, 1250, 1300° C. or any range therein, e.g., about 550-1200° C., about 600-1000° C., or about 800° C. In an additional embodiment, the thermal treatment of the SiC device is carried out for about 2-8 hours, e.g., about 2, 3, 4, 5, 6, 7, or 8 hours, e.g., about 4-6 hours. In one embodiment, the thermal treatment of the SiC device is carried out for about 5 hours.

In an additional embodiment, exposing the SiC device to nitrogen may include exposure to any form of nitrogen. The source of nitrogen can include, without limitations, a solid, liquid or gas source. In some embodiments, the step comprises exposing the SiC device to a nitrate compound, such as ammonium nitrate or sodium nitrate, e.g., at about room temperature. In another embodiment, exposing the SiC device to nitrogen comprises exposing the SiC device to NH₄OH or another ammonium compound, e.g., at about room temperature. In an additional embodiment, exposing the SiC device to nitrogen comprises exposing the SiC device to a nitrogen atmosphere, e.g., at about 900-1300° C.

In another embodiment, after exposing the SiC device to nitrogen to form silicon nitride on the SiC device surface at least 20% of the surface of the SiC device is coated in silicon nitride, e.g., about 20%-100%, e.g., at least 20%, 30%, 40%, 50%, 60%, 70%, 80%, or 90% or any range or value therein. Silicon nitride has a high-melting point solid and is relatively chemically inert and very hard (8.5 on the Mohs scale). Silicon nitride has better high temperature capabilities than most metals. Benefits include low density, high temperature strength, superior thermal shock resistance, excellent wear resistance, high hardness and toughness, resistance to mechanical fatigue and creep, and good oxidation resistance. Specifically, silicon nitride increases strength and provides a material that is wear-resistant and suitable for high-temperature applications such as, without limitation, bearings, cutting tools, turbine blades, ballistic protection, automotive manufacturing, electrical vehicle manufacturing, filters, metal purification systems, or medical devices. Additionally, silicon nitride exhibits increase porosity and yields high flexural strength, high fracture resistance, good creep resistance, high hardness, and excellent resistance to erosion. In addition, silicon nitride has thermomechanical and tribological properties and provides good performance in terms of hardness and fracture toughness, which are strictly required for high-load medical applications. Silicon nitride is non-ferrous, non-electromagnetic, and partially radiolucent that minimizes scatter and associated artifacts on computerized tomography (CT) and magnetic resonance imaging (MRI) in patients with silicon nitride implants.

In a further aspect, the present invention relates to a method of increasing strength of a SiC device, comprising: (i) exposing SiC particles to NaOH to create a silica gel layer; (ii) drying the SiC particles, e.g., in air, to dry the silica-gel layer; (iii) mixing the SiC particles with aluminium oxide and/or magnesium oxide and/or silicate mineral particles such as mullite, cordierite, cristobalite, or spodumene; (iv) preparing a SiC device using the mixture of step (iii), e.g., using a 3D printer or by pressing in a mold; and (v) thermally treating the SiC device at about 800-1400° C. for 4-24 hours.

SiC particles may be exposed to NaOH as described above. The mineral particles may be about 10-200 microns in diameter, e.g., about 50-100 microns, e.g., less than 200, 150, 100, or 75 microns in diameter. The SiC particles may be mixed with the aluminium oxide and/or magnesium oxide and/or minerals at about 5-25% by weight, e.g., about 15-20%. The SiC device may be prepared by any method described herein, such as 3D printing or powder metallurgy. The mixture may be pressed into a mode at about 250 MPa. In some embodiments, the SiC-mullite device may be heated to about 1400° C. and the SiC-cordierite and SiC-spodumene devises may be heated to about 1200° C.

Regarding bioactivity and biocompatibility, silicon nitride is able to accelerate bone repair and induce osseointegration. Moreover, silicon nitride demonstrates antibacterial ability and tissue innervation, which is essential for orthopedic implants. Specifically, the elution kinetics of N and Si species makes the surface environment of silicon nitride toxic to bacteria and nutritious to eukaryotic cells, as a function of pH.

In an additional embodiment, the SiC device is a medical device. A medical device is an instrument, apparatus, implement, machine, contrivance, implant, in vitro reagent, or other similar or related article, including a component part, or accessory which is: (a) recognized in the official National Formulary, or the United States Pharmacopoeia, or any supplement to them; (b) intended for use in the diagnosis of disease or other conditions, or in the cure, mitigation, treatment, or prevention of disease, in man or other animals, or (c) intended to affect the structure or any function of the body of man or other animals, and which does not achieve its primary intended purposes through chemical action within or on the body of man or other animals and which is not dependent upon being metabolized for the achievement of its primary intended purposes. In another embodiment, the SiC device is a medical screw, plate, stent, cage, rod, plug, pin, implant, mesh, or cable.

In another embodiment the SiC device is a ground-based imaging system device or a space-based imaging system device. A ground-based imaging device or a space-based imaging device is an instrument that gather scientific information to explore new planets, moons, and our solar system and beyond. These instruments measure space plasma, particles, ultraviolet lights (UVs), high and low energy and perform remote sensing.

In another embodiment, the SiC device is a lithography positioning tool or a stepper system device. A stepper is a device used in the manufacture of integrated circuits (ICs). It is an essential part of the process of photolithography, which creates millions of microscopic circuit elements on the surface of silicon wafers out of which chips are made. A lithography tool is typically used for printing semiconductor integrated circuits.

In an additional embodiment, the SiC device is a scanning mirror system device or optoelectronic device. Scanning mirrors are lightweight mirrors that are used for high-speed two-axis laser scanning systems. Optoelectronics (or optronics) is the study and application of electronic devices and systems that find, detect and control light, usually considered a sub-field of photonics. In this context, light often includes invisible forms of radiation such as gamma rays, X-rays, ultraviolet and infrared, in addition to visible light. Optoelectronic devices are electrical-to-optical or optical-to-electrical transducers, or instruments that use such devices in their operation.

In another embodiment, the SiC device is a ballistic protection device. Ballistic protection involves protection of body and eyes against projectiles of various shapes, sizes, and impact velocities. Ballistic protection concerns apparel, vests, armors, helmets, and structural reinforcement for vehicles as well. The woven, knitted, or nonwoven fabrics, laminates, and composites are used for ballistic protection. The type (knife, handgun, assault rifle bullet, high-velocity bullet) and level of the threat are considered in design and manufacturing of ballistic protective apparel. The structure of armors may include ceramic plates, special fibers/textile structures, laminated/coated textiles, and composites depending on these parameters. In addition, blunt impact protection could be imparted to armors by including shock-absorbing materials.

In additional embodiment, the SiC device is an energy or nuclear power device. An example of an energy or nuclear power device is a nuclear reactor, which is a device used to initiate and control a fission nuclear chain reaction or nuclear fusion reactions. Nuclear reactors are used at nuclear power plants for electricity generation and in nuclear marine propulsion.

In another embodiment, the SiC device is a component of an automotive device such as catalyst, brake, exhaust, or suspension. In an additional embodiment, the SiC device is a component in an electrical vehicle such as insulated gate bipolar transistor. An insulated gate bipolar transistor is a three-terminal power semiconductor device primarily forming an electronic switch. It was developed to combine high efficiency with fast switching. It consists of four alternating layers (P—N—P—N) that are controlled by a metal-oxide-semiconductor (MOS) gate structure. A SiC insulated gate bipolar transistor shows stronger competitiveness in high voltage, high temperature, and high-power fields. In another embodiment, the SiC device is a component of a filter device. The SiC filter can be resistance to molten iron erosion and corrosion. They effectively remove inclusions, reduce entrapped gas in the liquid metal and provide laminar flow, resulting in significantly cleaner filtered substrate.

In another aspect, the present invention relates to a method of implanting or attaching a SiC device, e.g., a medical device, to a subject in need thereof, comprising using the SiC device of the present invention. A subject in need thereof can be a person who has sustained an injury or has a disease involving the musculoskeletal system, specifically the bones, joints, ligaments, tendons, and muscles. Accidents, falls, sports-related occurrences, or other high-impact events frequently lead to these injuries. Orthopedic injury or disease involves wounds to the bones and supporting structures that cause pain and functional limitations. Minor fractures to complex fractures, dislocations, ligament rips, and severe soft tissue injuries can all result from these injuries. The causes of orthopedic trauma include, without limitation, accidents falls, sports injuries, work-related injuries, aging and osteoporosis. Treatment tries to recover the affected area's stability and function. Depending on the kind and extent of the injury, there are a variety of potential therapies, such as: (a) immobilization with casts, splints, or braces may promote proper bone healing for less severe injuries; (b) reducing fractures requiring realignment includes moving the bones back into the proper alignment, which is how alignment-required fractures are handled. During the healing phase, fixation techniques may hold the bones together, including screws, plates, and rods; and (c) external devices like pins, cables, or frames occasionally attach to the bones to give stability and speed healing. Implants are tools used to support and stabilize broken bones while they heal. The location, kind, and complexity of the fracture are just a few of the variables that affect the implant's decision. Typical orthopedic trauma implants include the following: screws and plates, which are used to join broken bones together and stabilize fractures. Typically, plates are attached to the outside of the bone, and screws hold the fragments in place; intramedullary rods, which are implants that are put into the center hollow of long bones to offer stability and support during the healing process; external fixators, which are devices that include inserting pins or cables through the skin into the bone. The pins are next linked to an outside frame to hold the bone fragments in place; internal nails, which are placed into the medullary canal of long bones, similarly to how rods are, and they help to stabilize the body internally and enable early patient movement; and bone grafts may be utilized when there has been severe bone loss or when a fracture is not healing. These grafts can be made of synthetic materials, a donor's organ, the patient's own body (auto-graft), or both. They aid in bone regeneration and repair.

In an additional aspect, the present invention relates to a method of stimulating innervation of tissue in a subject in need thereof, comprising contacting the tissue with the SiC device, e.g., a medical device, of the present invention. Innervation of tissue is a process by which nerves grow and form connections with target tissues or organs. Innervation promotes the development of tissues and organs, but also has a central role as a tool for their functional control and modulation. Innervation is needed, for example, for the function of cardiac, skeletal, or smooth muscle containing tissues (e.g., stomach or bladder). Innervation is also needed in tissue engineering for the proper functioning and integration of the implanted constructs with the host tissue. Silicon nitride promotes tissue innervation by promoting the healing of bone tissue but inhibiting the proliferation of bacteria due to the kinetics of N and Si species makes the surface environment of silicon nitride toxic to bacteria and nutritious to eukaryotic cells. As such, silicon nitride implants promote tissue fusion and the osseointegration of surrounding tissue.

The present invention is more particularly described in the following examples that are intended as illustrative only since numerous modifications and variations therein will be apparent to those skilled in the art.

### EXAMPLES

### Example 1: Orthopedic Screws

### Methods

SiC particles composed of 40% alpha SiC, 50% beta SiC and 10% amorphous SiC in weight ratio and with average size range of: 70% (40 μm), 15% (2 μm) and 15% (600 nm) were utilized to print SiC screws. All SiC particles were purchased from (US Research Nanomaterials, Houston, TX). The 40 μm SiC particles were treated with 15% NaOH/15 min prior to mixing with the smaller SiC particles. A powder bed binder ink jet 3D printer (ProJet460Plus, 3D System, USA) along with water-based ink was used for printing.

The AUTOCAD program was used to design a screw that mimics a commercially available titanium alloy ACL screw. See Table 1 below. The AUTOCAD design file was converted to STL format and fed to a 3DPrint software for conversion into a machine readable ZBD file. Performed at room temperature, the 3D printing process featured a printing speed of 2-4 layers per minute, and a layer thickness of 102 μm. See Table 1 below.

TABLE 1
Design of screws and compression cylinders

| Design parameter | Dimensions (mm) |
|---|---|
| Length | 24.89 |
| Inner diameter | 4.38 |
| Outer diameter | 9.14 |
| Thread separation | 0.51 |
| Thread length | 2.38 |
| Head diameter | 10.16 |

The initial SiC particle size range used for 3D printing includes particles in the size range 10 nm to 80 microns. To facilitate flow-ability and homogeneous layer dragging during printing, multi-modal particle size was used including 70% of the particles have average size 40 micron, 15% are 2 micron and 15% are 0.6 micron. The selection of the particle sizes and the corresponding ratios were based on the size of the pores observed by SEM on the SiC discs made of <80 nm and 40 μm particles at a ratio 1:15. The printed SiC objects with the multimodal particle size range showed better structural integrity and a smooth surface finish than the ones printed using 40 μm particles or a mixture of 40 micron and <0.08 micron at a ratio 15:1.

The SiC particles in the size range 40 micron are pre-immersed in 20% NaOH for 15 min to create a silica gel layer that mediates the bonding between SiC particles upon spraying with water-based binder in a powder bed printer. The thickness of the silica layer can be increased by increasing the NaOH concentration and immersion duration. After NaOH treatment, the SiC particles were washed, dried, separated and sifted. The dried pre-treated SiC particles were mixed with 15% particles with average size 2 micron and 15% particles with average particle size 600 micron in an alumina ball mill.

Heat treatment of the SiC screw in the temperature range 550-1300° C. crystallizes the silica gel into cristobalite. At the low temperature end quartz is formed in addition to cristobalite. The silica layer on SiC can be converted into silicon nitride (Si₃N₄) by heating in nitrogen atmosphere or by immersing the screws in ammonium hydroxide solution. The formation of Si₃N₄ enhances the mechanical and biocompatibility properties of the SiC screws.

To print the SiC screws, first the digital image of the threaded screws was designed using Creo parametric 3D modeling software. The digital image was then converted to a 3D part by converting it to an STL file. The SiC powders were printed in layers with an average thickness of 102 μm. Commercially available aqueous binder solution was used to spray the particles during printing, which resulted in rehydration of the dried silica gel layer on the SiC particles. Rehydration of the silica gel promotes particles bonding via the condensation reaction of the silanol groups on adjacent particles. See FIG. 15.

In total, nine screw models (designs) were printed. The main alterations made in the screw design were adjusting the screw bit head type, size, and sharpness of the threads, and adding a hollowness aspect to the screw design.

Heat treating 3D printed SiC at 900° C. for 2 h resulted in a strength of 0.3 MPa. Subsequently, impregnation in NaOH and applying a second heat treatment at 900° C. resulted in a 13-fold increase in the strength.

After printing, the screws were removed and heated at 650° C. for 5 hrs. Loose SiC particles present on the printed screws were removed by airbrushing. After cleaning, the screws were subjected to additional post-processing protocols including immersion in an alkali solution and thermal treatments. See Table 2 below.

TABLE 2
Post-processing protocols applied to the 3D printed
SiC screws after 650° C./5 h treatment

| Protocol # | Post-processing |
|---|---|
| 1 | 15% NaOH/15 mins + air dry + |
| | 550° C./6 h + 1200° C./24 h |
| 2 | 15% NaOH/15 mins + air dry + |
| | 550° C./6 h + 1150° C./24 h, cool |
| | to room temperature, 20% NAOH/30 mins + |
| | 5N NH₄OH/24 h + 1200° C./2 h |

Subjecting the 3D printed SiC to a thermal treatment at 650° C. prior followed by impregnation in NaOH and then a second heat treatment at 800° C. resulted in a strength which is 20 times greater than subjecting the samples to one step heat treatment at 900° C. for 2 h. The significant increase in the mechanical strength is associated with the increased formation of the silica gel on the pore surfaces of the 3D printed SiC treated at 650° C. At 650° C., the surface of SiC is covered with amorphous silica layer and quartz which can easily react with the NaOH. Treating the discs at 900° C. prior to impregnation in NaOH resulted in formation of mature cristobalite crystals that is less reactive to NaOH.

Sample impregnated in 15% NaOH for 15 mins and heat treated at 550° C. for 6 hours and 1100° C. for 24 hours at 1° C./min, showed the highest compressive strength of 40.25±2.5. MPa. Comparable compressive strength 36.76±5.04 MPa was observed when the treatment temperature was increased to 1200° C. for 24 hrs

Measurements of porosity and density: A density determination kit (Ohaus, Parsippany, NJ) and digital scale (Ohaus, Parsippany, NJ) were used to measure the density and apparent porosity. A thermometer was used to measure the temperature and determine the water density value. 3D printed SiC samples were weighed in air and in water. The weight of the water inside the pores was calculated as the difference between the weight of the sample before and after immersion in water. According to the principle of Archimedes, the density (D, g·cm⁻³) and apparent open porosity percent (Pₐ) are calculated using equations (1) and (2), respectively.

$$D = \frac{{M}_{a}}{{M}_{a} - {M}_{L}} ( {D}_{L} - {D}_{a} ) + {D}_{a} \qquad ( 1 )$$

Where Mₐ is the mass of the dry sample in air (g), M_{L} is the mass of the sample in water (g), D_{L} is the density of the DI water (0.997 g/cm³ at 24° C.), Dₐ is the density of the air (0.0012 g/cm³), and where Mₛ is the mass of the water-saturated sample in air (g).

The apparent open porosity was calculated using Eq. (2):

$${P}_{a} = \frac{{M}_{s} - {M}_{a}}{{M}_{a} - {M}_{L}} \times 100 \% \qquad ( 2 )$$

Mechanical Test: 3D printed SiC threaded cylinders were designed and printed for compression test according to ASTM standard (C1424-99) with an inner diameter of 10 mm and a total length of 20 mm maintaining the 1:2 ratio. On both ends of the cylinder the inner diameter of 10 mm was CAD extruded. The threaded cylinders were 3D printed following the same procedures mentioned above and were subjected separately to post processing treatment as described in Table 2. The mechanical properties of thermally treated 3D printed SiC cylinders (n=5) were evaluated using an Instron Machine Model 5582 at room temperature and in air. The 3D printed SiC samples were loaded uniaxially at a rate of 0.020 in/min until failure. The data for the load vs. displacement were recorded and the modulus of elasticity and compressive strength were calculated. The strain was calculated by dividing the displacement by the length of the sample. The stress was calculated by dividing the load by the cross-sectional area of the sample. The compression modulus of elasticity was calculated from the slope of the linear trend derived from the stress vs. strain curve.

Scanning Electron Microscopy-Energy Dispersive X-Ray analysis (SEM-EDX) analysis: The surface morphology of the intact SiC screws as well as the fracture surface were analyzed using the SEM-EDX analysis. Samples were first sputter coated with a 15 nm thin gold layer and analyzed in a secondary electron mode using a SEM JEOL 6480 microscope. EDX elemental composition was analyzed at representative material surface locations.

X-ray diffraction (XRD, X′ PertPRO): The 3D printed SiC screws thermally treated according to protocol #2 showed the highest mechanical strength, therefore were separately ground into powder and the phase composition was analyzed by XRD, X′ PertPRO analysis with Ni-filtered Cu K (alpha) radiation at 45 kV and 40 mA.

SiC screw surface analyses and silica release: A SBF was prepared with ionic concentrations and pH mimicking human plasma. The pH of the SBF was measured to ascertain it is similar to that of healthy human plasma. 3D printed screws were immersed in 50 mL of SBF for 3, 5 and 11 days at 37° C. and maintained under continuous agitation (80 rpm) using an orbital shaker. The SBF was exchanged every 3 days. After immersion, the screws were removed from the SBF and washed in DI water for 3 minutes to remove any loose salt build up during immersion. The formation of hydroxyapatite surface layer on the 3D printed SiC screws was observed by Fourier transform infrared spectroscopy (FTIR; Nicolet 6700 Thermo-Nicolet, Madison, WI) and SEM analyses.

The outer and fracture surface chemistry of the SiC orthopedic screws were analyzed before and after immersion in SBF by FTIR in the diffuse reflectance mode (DRIFT). Spectra were small fragments of the 3D printed SiC screw samples that were fractured while ensuring that the surface of the samples remained intact for FTIR analysis to be done. Fine IR-grade Potassium bromide (KBr) was used to collect the background. Sample fragments of the screw were loaded into the sample holder on top of the KBr, ensuring that the surface was flat and leveled. The sample spectrum was collected after 200 scans at 4 cm⁻¹ resolution. Sample spectra were then refined using the auto baseline correction feature, smoothed, and converted to Kubelka-Munk using Omnic software program available in the analyzer. The spectra were deconvoluted in Origin program to resolve broad peaks and identify bands in the wavenumber range characteristic to hydroxyapatite.

Inductively Coupled Plasma-optical Emission Spectrometry (ICP-OES) concentration measurements of Silica release from 3D printed SiC screws: 3D printed SiC orthopedic screws (n=3) subjected to post processing protocol #1 were immersed separately in 10 ml PBS and incubated for 24 hrs at 37° C. The immersing solution was collected, filtered and used to measure the ionic concentration and to study cell response. For measurement of ionic concentration, the PBS samples preincubated with the 3D printed SiC orthopedic screws (n=3) were subjected to digestion process prior to ICP-EOS measurement. Approximately 100 mg of the solution sample was weighed directly into a Teflon microwave vessel. 1 mL sulfuric acid, 1 mL nitric acid and 0.5 mL of hydrofluoric acid were added to each vessel, a blank for the standards is prepared in the same manner. Samples were digested in the microwave. The temperature was ramped to 200° C. in 15 minutes and maintained at 200° C. for another 15 minutes then cooled and diluted to a final volume of 10 mL using E-pure water. There was no need for sample filtration. The ionic concentrations of Si, and Na in the collected SiC extract media (n=3) were analyzed by ICP-OES (ICO-OES; Perkin Elemer 5300 DV ICP-OES). Si was measured at the wavelength 288.158 nm axial and Na was measured at the wavelength 588.995 nm radial. A two-point calibration (0.1 ppm and 4 ppm) that is linear to 16 ppm in-solution was used to construct a calibration curve for the analyte of interest and calculate the concentration of the samples (ppm). Parallel experiments following the same procedures were employed to measure Titanium ion concentration in PBS solution incubated with Ti-6Al-4V discs (1 mm×10 mm dia) for 24 hours.

Biological effects of released Si ions and SiC screws on Macrophages, Neurons and Osteoblasts: The PBS extract of the 3D printed SiC orthopedic screws and Ti-6Al-4V were added separately to tissue culture medium (TCM) incubated with J774 macrophages. Briefly, confluent J774 cells were first activated toward an M1-like phenotype through sequential incubations with IFNγ (100 IU/ml, 6 hrs) and lipopolysaccharide (LPS) (20 ng/ml, 24 hrs). Following IFNγ activation and PBS wash, cells were incubated with LPS and various dilutions of the ceramic extract (in PBS) combined with 10×DMEM and water to final 1×DMEM concentration and final PBS/TCM volume ratio of ½ (50:50), ¼ (25:75), and 1/10 (10:90), respectively. Each sample was assessed in triplicate in multiple independent experiments.

Following a 24 hr incubation under 5% CO₂; 37° C., >90% humidity, J774 cells were stained with both Hoechst 33342, a cell membrane permeant fluorophore that combines with DNA and render nuclei fluorescent at 462 nm following excitation at 350 nm and propidium iodide, and a cell membrane impermeant fluorophore that combines with DNA and render nuclei fluorescent at 617 nm following excitation at 535 nm. Fluorescence emitted at both 462 and 617 nm was determined using a ID5 plate reader (Molecular Device) and the ratio PI/Hoechst (no unit) that defines the proportion of cells with degraded membrane, a marker of dying/dead cells, was derived.

Reactive oxygen species (ROS) activity, a measure of cell stress, was determined in separate experiments using the cell membrane permeant fluorescent ROS marker H2DCFDA (Ex/Em: 492/517 nm). Data were collected as fluorescent unit (FU).

Immune activities of J774 macrophages incubated separately with SiC and Ti₆Al₄V dissolution products were determined using supernatants collected following a 24 hr incubation and assayed by sandwich ELISA for both secreted TNFα and shed IL6R. Samples were run along with standard curves. Using the standard curves, concentrations of secreted TNFα and shed IL6R were determined and expressed in pg/ml.

All experiments relating to animals were performed in accordance with protocols approved by the Institutional Animal Care and Use Committee (IACUC) of the University of North Carolina Charlotte. Embryos of E15 pregnant Sprague Dawley rat was used as a source of embryonic DRG neurons. Embryos were dissected under a stereoscopic dissection microscope (Nikon SMZ745T, Japan) and DRG cells collected as reported earlier. The DRG culture medium consisted of neurobasal medium supplemented with 1% 100× P/S, 1% glucose, 100× 2% glutamate, 2% B27 supplement, and 20 ng/mL NGF. Fluorodeoxyuridine (FUDR, 13 μg/mL) was added in culture media to minimize the possible growth of non-neuronal cells. Neuron cultures were maintained by replenishing half of the media every 72 hrs.

3D printed SiC discs were placed on a 12-well plate and coated with poly-L-lysine/Laminin for 4 hours at room temperature. Then they were washed with PBS thrice followed by immersion in the culture medium for 4 h. The discs (n=3) were placed separately in 3 ml culture dishes and seeded with 500 L cells suspension (1×10⁵ cells/mL) dropwise. The cell-loaded discs were incubated initially for 30 min in κ% CO₂ at 37° C. to allow for cell adhesion. Thereafter, 2.5 mL additional medium was added into the well and kept in culture condition for 7 days. The medium was replenished every 72 hours. The cells were stained with Calcein AM dye (Corning, USA) and imaged by florescence microscope on day 7. SiC samples incubated with neurons for 7 days were fixed with 4% glutaraldehyde for 1 h and dehydrated in ethanol (25, 50, 75 and 100%, respectively; 20 min each) for SEM microscopy.

Fully differentiated primary murine osteoblasts were isolated from whole calvaria of two to three-day old murine neonates through six sequential 15-min trypsin/collagenase P digestions and the cells were maintained in DMEM supplement with 10% FBS and 1% penicillin/streptomycin at 37° C. in a 5% CO₂ atmosphere. At 24 h, primary osteoblasts were plated in 6-well plates at a density of 2×10⁵ cells per well and differentiated in αMEM supplemented with 10% FBS, 0.1 M ascorbic acid, 1 M β-glycerol phosphate, and 100 U/mL penicillin/100 μg/mL streptomycin at 37° C. in a 5% CO₂ atmosphere. The differentiation media was changed every other day until the experiment at day 10, and the presence of mature osteoblasts was confirmed using an alkaline phosphatase staining kit (Abcam). 3D printed SiC screws (n=3) were sterilized by immersion in pure ethanol for 12-hr and dried under the laminar flow hood. Cells were trypsinized upon 70-80% confluency, resuspended in TCM. Osteoblasts (5×10⁴ cell) were seeded on the surface of screws and covered with complete tissue culture medium. After 2 days, the media was exchanged and supplemented with 3 mM β-glycerol phosphate, dexamethasone (10⁻⁸ M) and ascorbic acid (50 μg/ml). The same exchange was repeated every 2-3 days. After 6 days, the samples were fixed in glutaraldehyde, dehydrated in ethanol, and coated with gold. Cell adhesion and spreading were analyzed by electron microscope.

Statistical analysis: Data are presented as mean±SEM. Graph and statistical testing were conducted using Prism 10 (Graph-pad Inc). Conditions were compared using one-way ANOVAs and post-hoc tests with a priori, p<0.05 considered statistically significant.

### Results

Immersion of the SiC screws in simulated body fluids resulted in deposition of a calcium phosphate layer similar to the mineral phase of bone, indicating bioactivity. Furthermore, the SiC screws with the surface composition made of silica or Si₃N₄ enhanced primary bone cell attachment, spreading, proliferation, differentiation, and production of mineralized extracellular matrix.

Seeding primary neuron cells on SiC screws resulted in growth of the neuron cells and synthesis of long axons directly on the material surface, which indicates that the SiC screws can stimulate bone formation and innervation.

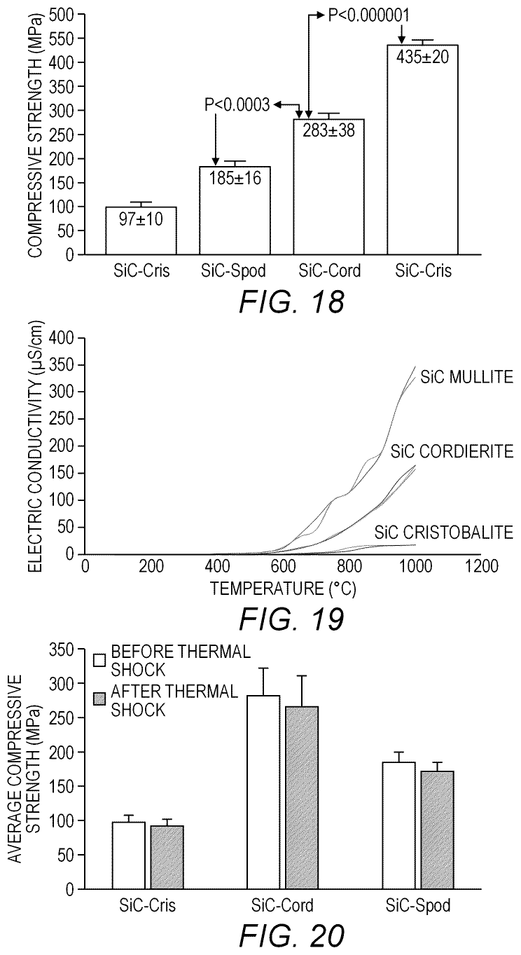

Moreover, addition of silicate mineral particles such as mullite, cordierite, cristobalite, or spodumene to the SiC particles increased density and mechanical properties. In these studies, cordierite or spodumene particles (<75 microns) were added at 20% by weight to SiC particles to prepare composites. Aluminum oxide was added at 15% by weight to form mullite. FIG. 18 and Table 3 show the mechanical properties of SiC-mineral composites. Table 4 shows the thermal properties of SiC-mineral composites. FIG. 19 shows the electrical properties of SiC-mineral composites, including electrical resistivity of 7.82×10⁹ and 2.785×10⁸ Ω·cm. FIG. 20 shows the average compressive strength for samples before and after water quenching of samples from 1200° C.

TABLE 3
Mechanical properties of SiC-mineral composites

| Property | Unit | SiC-Cord | SiC-Mullite |
|---|---|---|---|
| Young's modulus (E) | GPa | 240 | 377.9 ± 22.7 |
| Vicker's hardness | GPa | 8.12 | 4.65 ± 0.8 |
| Density (ρ) | g/cm³ | 2.74 | 2.26 |
| Compression strength | MPa | 282 | 434 ± 20 |

TABLE 4
Mechanical properties of SiC-mineral composites

| Property | Unit | SiC-Cord | SiC-Mullite |
|---|---|---|---|
| CTE (α) | 10⁻⁶/K | 0.4 × 10⁻⁶/ | 1 × 10⁻⁶/ |
| | | K at 372 K | K at 372 K |
| Thermal conductivity | W/m · K | 6.737 | 14.45 |
| (k@25° C.) | | | |
| Thermal diffusivity | 10⁻⁶ m²/s | 5.483 | 9.98 |
| Specific heat capacity | J/g · K | 0.574 | 0.445 |

Cell culture studies of SiC with each one of these minerals showed biocompatibility as indicated by bone cell adhesion, spreading, proliferation and differentiation on the surface of the material. Immersion of SiC-silicate mineral composites in simulated body fluid showed formation of calcified nodules with composition similar to the mineral phase of bone, indicating bioactivity.

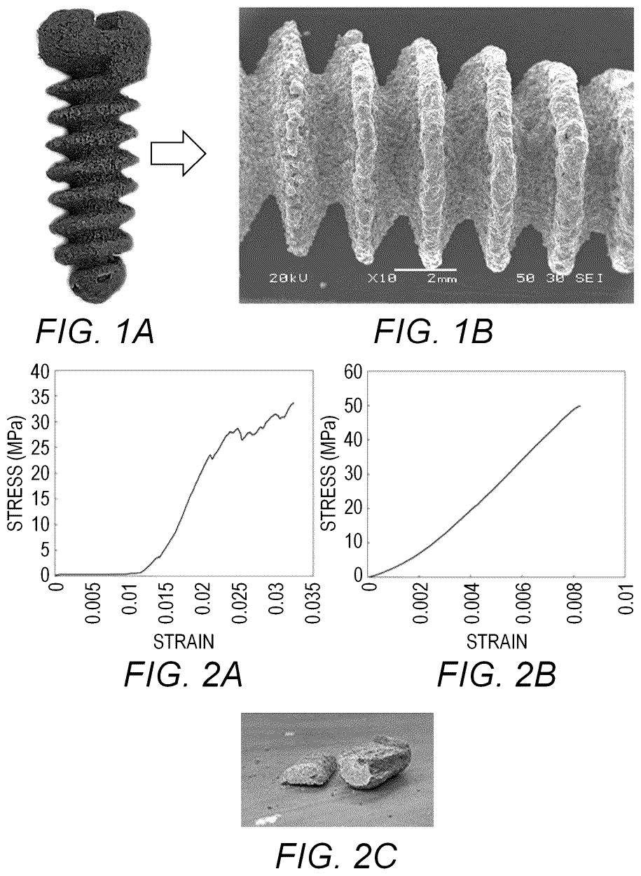

Additionally, the SiC-mineral as well as the SiC-silica and SiC—Si₃N₄ composites can be used in dense or porous forms as load-bearing implants and/or fixation devices. Alternatively, they can be used in a porous form for delivery of cells and biological molecules and as a coating on metallic implant to minimize corrosion and enhance tissue integration. SiC Screw Density, Porosity and Mechanical Properties: 3D printed bioactive SiC orthopedic screws (See FIGS. 1A-1B) and cylinders were used for mechanical testing after thermal treatment according to protocol number #2. See Table 2 above. The diameter shrinkage was 6.2±0.71%.

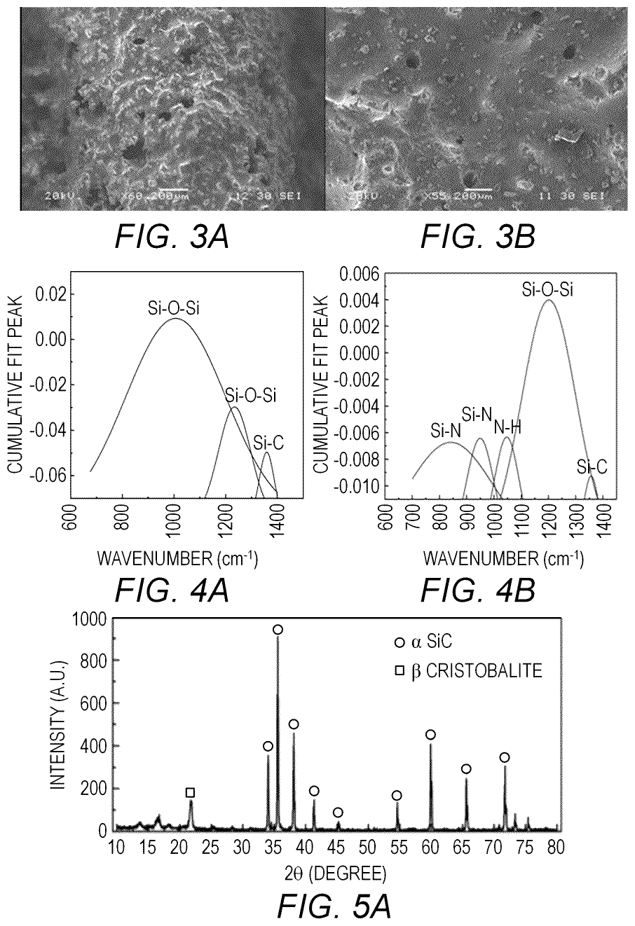

Protocol #2-generated samples had relatively lower porosity and higher mechanical properties compared to samples treated according to protocol #1. However, regardless of post-processing protocol used, sample densities were similar most probably because of increased cristobalite formation. Indeed, density of cristobalite is significantly lower than that of silicon carbide material which decreases the overall density. It is noticeable that load-displacement curves for samples treated according to protocol #2 showed an elastic behavior associated with a sudden rupture, as generally recorded for dense ceramics. See FIGS. 2A-2C. After the test, the exterior surface of the sample showed macro-cracks parallel to the direction of solicitation. SEM analysis indicated that the smoothness of the elastic behavior in the stress-strain curve is attributed to the strong fusion of the particles entrapping closed pores within the dense 3D printed SiC—SiO₂ composite. See FIGS. 3A-3B.

Deconvolution of the FTIR spectrum of the fracture surface of 3D printed SiC samples subjected to post processing treatment according to protocol #1 showed evidence of the formation of SiO₂ in addition to SiC. The band at 1005 cm⁻¹ is assigned to Si—O—Si. See Table 5 below.

TABLE 5
Density, porosity, compression strength, and Young's
modulus of 3D printed threaded SiC cylinders subjected
to two different post-processing protocols

| | | | Compression | Young's |
|---|---|---|---|---|
| Protocol | Avg density | Porosity | strength | modulus |
| # | (gcm⁻³) | (%) | (MPa) | (GPa) |
| 1 | 1.94 ± 0.12 | 28.4 ± 7.7 | 28.1 ± 3.1 | 2.0 ± 0.5 |
| 2 | 1.94 ± 0.10 | 23.1 ± 8.4 | 39.9 ± 8.3 | 4.3 ± 1.2 |

This band disappeared in the sample treated according to protocol #2 and another low intensity band at 1050 cm⁻¹ is present which is characteristic of Si—N. The band at 1234 cm⁻¹ in the spectrum of the sample treated according to protocol #1 is due to the Si—O—Si, this band significantly shifted to 1203 cm⁻¹ in the spectrum of sample treated according to protocol #2. The deconvoluted spectrum of the samples treated according to protocol #2 showed bands at 840, 956, and 1050 cm⁻¹ which are attributed to the three components (ν₁-ν₃) Si—N bond. See FIG. 4B. The decomposition of the Si—N stretching mode into three components has been reported for both silicon-rich and stoichiometric silicon nitride films. The peak at 1,050 cm⁻¹ could be the stretching vibration N—H bond. The intense band at 1203 cm⁻¹ could be attributed to the Si—O—Si symmetric stretching vibration as well as the N—H wagging mode.

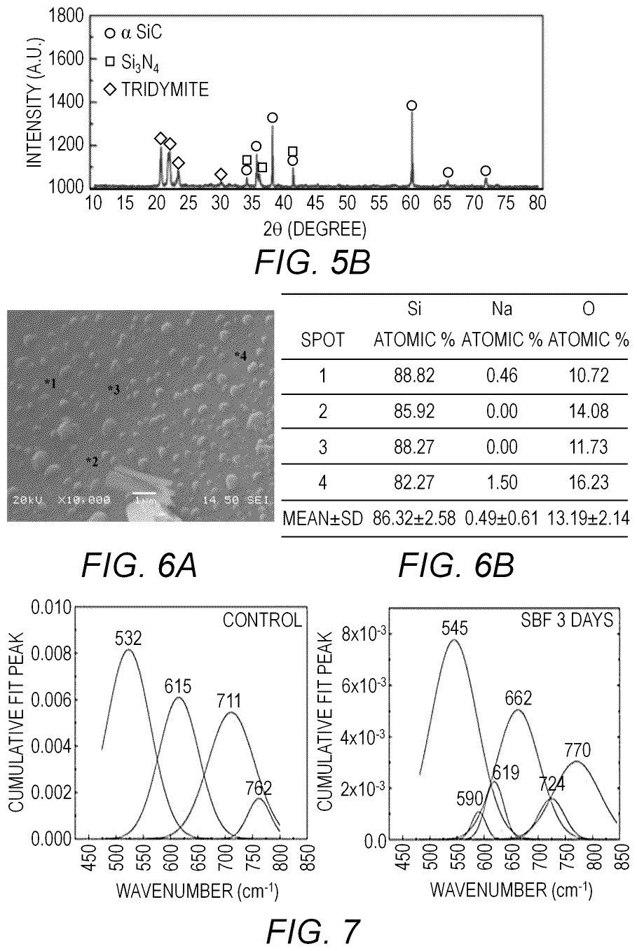

The XRD analysis demonstrated the characteristic peaks for α-SiC phase, cristobalite (SiO₂) solid solution for samples treated according to protocol #1. See FIGS. 5A-5B. The XRD analysis also showed characteristic signals for Si₃N₄ in addition to characteristic peaks for α-SiC phase, cristobalite (SiO₂) solid solution. See FIGS. 6A-6B.

Furthermore, the XRD analysis of the samples treated according to protocol #1 displayed the characteristic peaks of α SiC and β cristobalite solid solution. See FIG. 5A. XRD of samples treated according to protocol #2 presented signals characteristic of silicon nitride in addition to alpha SiC and tridymite. In association with the formation of silicon nitride, it was observed that a change in the intensity of the peak at 20=35.6º may likely be due to the decomposition of the SiOC and the formation of SINC. See FIG. 5B.

The SEM-EDX analysis of 3D printed samples heated at 550° C. for 20 min showed the formation of a silica layer with a homogenous distribution of silica droplets. Sec FIG. 6A. Moreover, EDX analysis defined the silica layer composition a (86.3±2.6%) Si and (13.2±2.1%) oxygen that it is highly oxygen deficient silicon oxide. (see Table, See FIG. 6B). The formation of the silica surface layer facilitated the deposition of a hydroxyapatite layer on the material surface upon immersion in SBF.

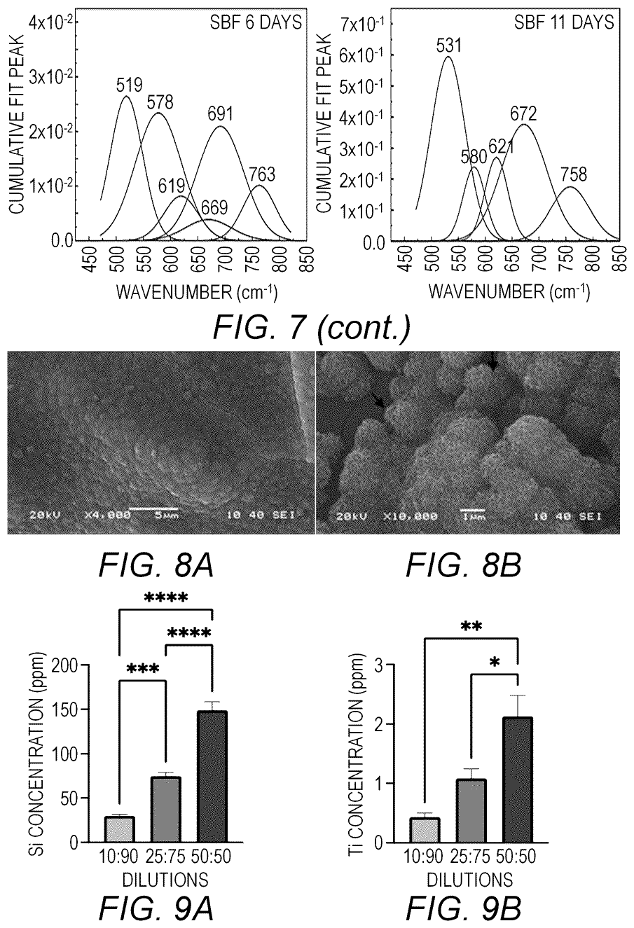

Immersion and SiC deposit and Si ion release in PBS: The impact of immersion time in SBF on the formation and maturation of a bioactive hydroxyapatite surface layer on the 3D printed SiC orthopedic screws was analyzed by FTIR. The deconvoluted FTIR peaks from the original broad peak domain of approximately 470-850 cm⁻¹ are presented. See FIG. 7. Doublet peaks at 590 and 619 cm⁻¹ representative of orthophosphate P—O bonds appeared after 3 days of immersion. The relative intensity of these peaks was equal at 580 and 621 cm⁻¹ following a 11-day immersion suggesting the formation of mature hydroxyapatite. Indeed, these two bands were not observed in control 3D printed SiC samples prior to immersion in SBF.

SEM analysis shows the precipitation of the calcium deficient hydroxyapatite layer onto the surface of 3D printed screws. Furthermore, the EDX analysis determined the presence of a 1.3±0.2 Ca/P ratio. See FIGS. 8A-8B.

The concentration of Si in the PBS incubated with the 3D printed screws (n=3) for 24 hours was 298±18 ppm. No measurable change was observed in the sodium ion concentration in the PBS incubated with the 3D printed SiC orthopedic screws. The titanium ion concertation in PBS incubated with Ti-6Al-4V discs (n=3) for 24 hours was 4.3±0.70 ppb.

Effects of increasing concentrations of released ions in PBS following incubation of either SiC screws or Ti-6Al-4V discs on macrophages were assessed, as discussed in further detail below. Specifically, the effects of three increasing silica and titania concentrations were investigated. Sec FIGS. 9A-9B.

SiC Samples Biological Activities on Macrophages, Neurons, and Osteoblasts:

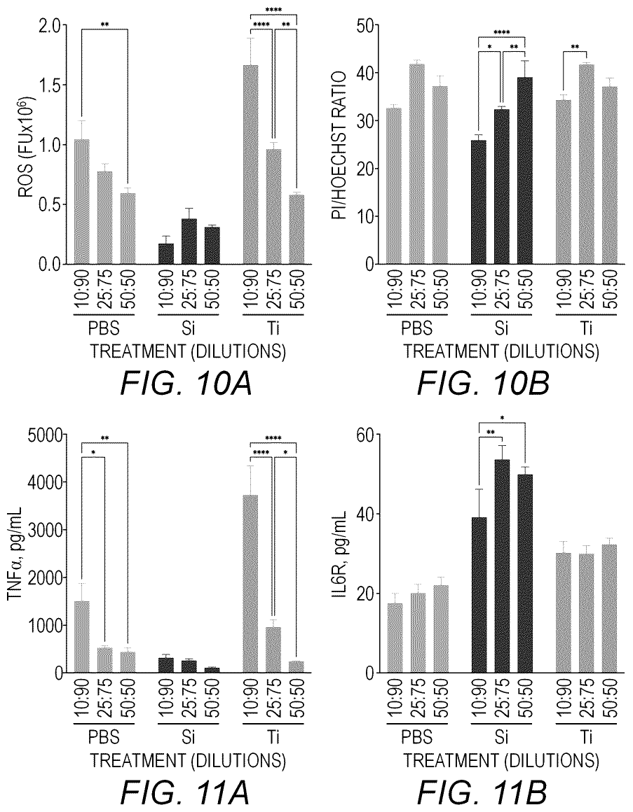

SiC dissolution products significantly suppressed reactive oxygen species, regardless of the dilutions tested, in comparison to medium treated with control PBS alone or with titanium dissolution products. Titanium dissolution products promoted reactive oxygen species expression at low concentrations. See FIG. 10A. Macrophage cell death, as determined by PI/Hoechst ratio, tended to be similar to that generated by PBS alone although SiC dissolution products led to a dose dependent increase also observed in the presence of titanium dissolution products. See FIG. 10B.

The J774 macrophages incubated with SiC dissolution products secreted significantly less TNFα in comparison with macrophages incubated with medium diluted with control PBS alone. See FIG. 11A. In contrast, J774 macrophages secreted significantly more TNFα following incubation with titanium dissolution products, especially at a 10:90 (titanium dissolution product to cell culture media) dilution. See FIG. 11A. In contrast, IL6R was shed at higher concentrations by macrophages following incubation with SiC dissolution products, whereas treatment with titanium dissolution products had no effects on sIL6R J774 macrophage supernatant concentrations. See FIG. 11B.

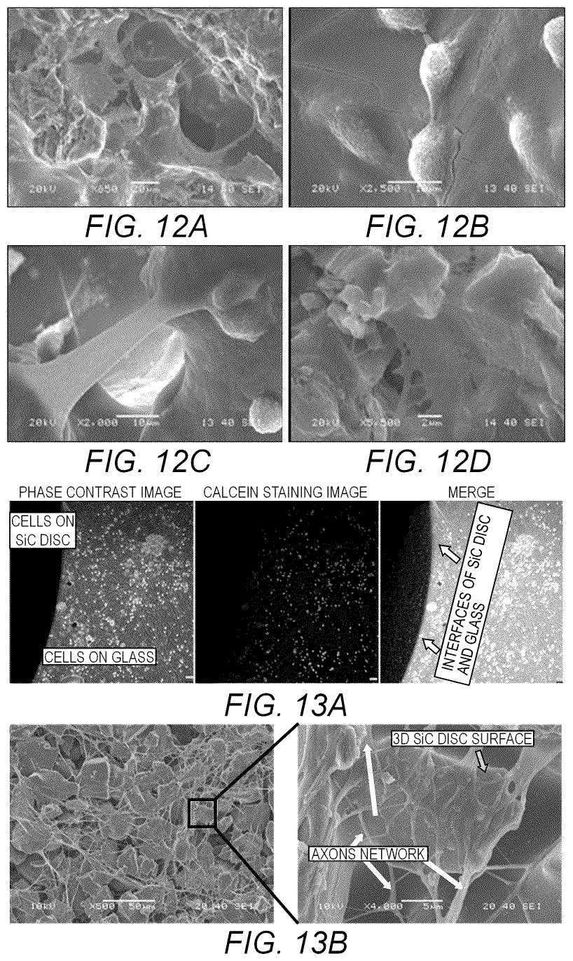

Furthermore, following a 6-day incubation, bone cells were attached and spreading onto the surface of the 3D printed screws supporting tissue integration. See FIGS. 12A-12D. The surface of the material was covered with layers of extracellular matrix that filled the screw pores.

Regarding the effect of SiC on neuronal growth, primary DRG neurons have an active metabolism when cultured onto SiC disks. See FIG. 13A. Moreover, increased growth was noted on the surface of porous 3D printed SiC disc as well as around the edge of the disc in contact with the culture dish. Importantly, a complex axonal network developed onto SiC surfaces and extended outwards. Sec FIG. 13B.

### Discussion

The results detail a new method for 3D printing of SiC ceramic orthopedic screws via surface activation. Chemical oxidation of SiC activated the surface of the material and enabled 3D printing of orthopedic screws in a powder bed binder jet printer. Post-processing impregnation of the 3D printed SiC screws with NaOH and/or NH₄OH prior to thermal treatment facilitated the mechanical strength due to further silica crystallization and/or partial conversion of the silicon oxide into silicon nitride; respectively. The new method for 3D printing of ceramic via surface activation differs from all prior processes and additive manufacturing research.

Specifically, rather than rely on high temperatures and pressures within a mold; or a polymer binder that serves as the preliminary support structure until it is removed by sintering, this novel approach is based on a chemical oxidation of the SiC surface using an alkali solution prior to printing. This produces a silicate film that enables bonding of SiC particles in a 3D printer at room temperature. This represents a transformative step forward in ceramic additive manufacturing that is applicable not only to SiC but can also be extended to other material systems.

Besides enhancing the 3D printing of SiC, the silica surface coating promoted the bioactivity property of SiC and modulated the immune response. Indeed, FTIR and SEM-EDX analyses demonstrated the formation of a hydroxyapatite layer on the 3D printed SiC upon immersion in SBF for 3 days. Furthermore, osteoblasts adhered to the 3D printed screws and produced extracellular matrix that covered the outer surface and filled the pores of the 3D printed screws. Moreover, neurons also adhered to the SiC scaffold and the adhesion was associated with the generation of an extensive network of axons confirming SiC scaffold support of neuron function. The 3D printed SiC promotion of osteoblast and neuron adhesion and function highlight the material potential in promoting bone remodeling/growth by osteoblasts along with innervation of new bone at the implantation bed.

Most importantly, silicate ions were the only ions released by the 3D printed SiC scaffold immersed in physiological solution as indicated by ICP-EOS analysis. Interestingly, increasing SiC ion concentrations in culture medium significantly downregulated features associated with M1 macrophages, i.e., TNFα secretions and ROS expression were reduced, whereas IL6R shedding increased. In contrast, titanium alloy released ions that promoted both macrophage TNFα secretions and ROS expression, both markers of proinflammatory M1 macrophages. These results highlight the role of dissolved silica ions in modulating macrophage phenotype and the associated inflammatory microenvironment to favor wound healing and regeneration of healthy bone.

Chemically, the Si—C bond is directional due to the lower electronegativity of Si (1.8) compared to that of C (2.5). The oxidation process commences with the introduction of NaOH to the SiC surface, where a preferential oxidation of the positively charged Si, with the negatively charged-OH takes place. The oxidation species generated during the NaOH-induced opening of the original Si—C bonds include Si(OH)₂, Si(OH)₃, and Si(OH)₄. Silanol groups (Si—OH) thermodynamically favor the re-polymerization via a condensation reaction at room temperature, resulting in the formation of a silica (SiO₂) gel layer. See FIGS. 13A-13B.

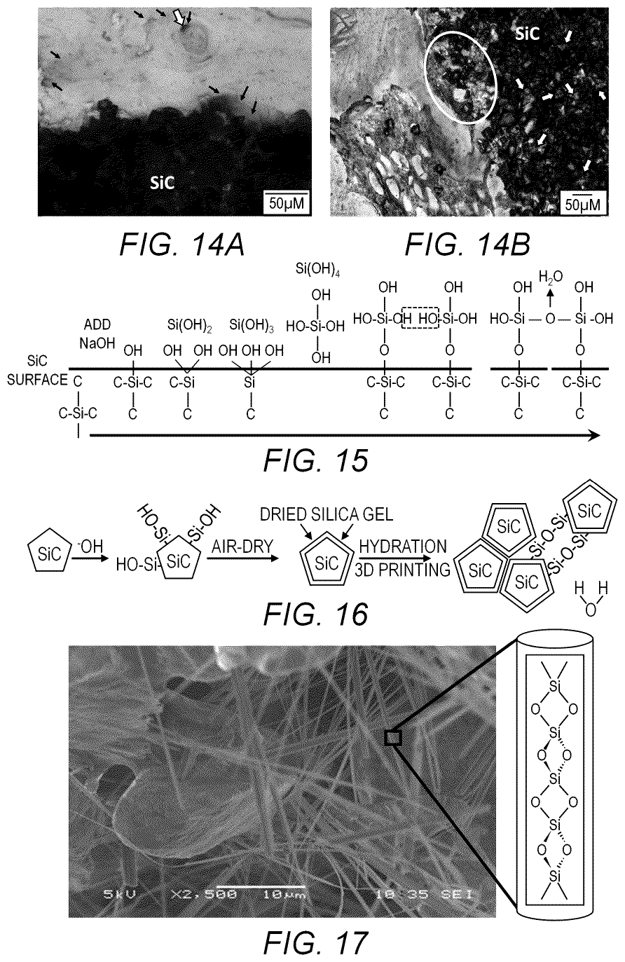

Moreover, the OH functional groups on the surface of ceramic form a microscale gel layer that can be airdried. The high charge prevented agglomeration of ceramic particles, enhanced packing and facilitated consistency of dragged ceramic layer during 3D printing. In the printing process, a water-based binder hydrates the surface-active silica layer facilitating SiC particles bonding via Si—O—Si bonds. See FIGS. 14A-14B.

Subjecting the 3D printed SiC to alternating thermal and alkali impregnation treatments enabled control of the thickness of silica bonding zone which in turn controlled the mechanical properties. This approach eliminates both the risk of distortion during printing and of collapse during de-binding. The post processing thermal treatment was associated with minimal shrinkage due mainly to the following two reasons. First, the formation of a homogenous silica layer on the SiC particles served as a shield minimizing oxygen diffusion during thermal treatment that otherwise would lead to significant oxidation of SiC. Second, due to the thermal stability of SiC, the dimensional changes during thermal treatment are confined to the micro scale silica layer that mediates the bond between SiC particles.

The compression strength and modulus of elasticity of the 3D printed screws are within the range of healthy human trabecular bone. Sec Table 2 above. Indeed, the modulus of healthy human trabecular bone ranges between 10 MPa and 3 GPa, whereas strength, which is linearly and strongly correlated with modulus, is generally two orders of magnitude smaller, in the range 0.1-30 MPa. The matching in mechanical properties between 3D printed SiC screws and trabecular bone is a major advantage over metallic and polymeric implants used for orthopedic applications like anterior cruciate ligament (ACL) surgery; one of the most prevalent knee injuries. Typically, ACL reconstruction uses graft fixation devices generated using either titanium alloy (Ti₆Al₄V) or a degradable polylactic acid polymer (PLA). Despite their biocompatibility, both Ti₆Al₄V and PLA fail to stimulate osteogenic differentiation of stem cells, vascularization, innervation, or mineralization during new tissue formation. Moreover, the relatively high stiffness of titanium alloy induces stress shielding in the adjacent bone, resulting in reduced bone density. Long-term studies have revealed the release of metal ions into the bloodstream, highlighting a risk of toxicity of those corrosion byproducts to filtering organs. Moreover, the acidic byproducts generated by PLA accumulate at the implantation site, where they can cause cell damage, inflammation, and foreign body reactions leading in some cases to osteolysis or cyst formation. Other notable limitations of Ti₆Al₄V and PLA devices including slow graft remodeling and incomplete ossification may be alleviated using the 3D printed bioactive SiC device of the invention.

Mechanically, existing biocompatible materials used for bone implants are selected based on their strength such as titanium alloy or structure characteristics that mimics the mineral phase of bone such as hydroxyapatite or tricalcium phosphate. However, these materials are unable to promote bone innervation, vascularization, or an immune environment favorable to multi-tissue regeneration. Indeed, these materials are primarily tailored to mimic inorganic bone mineralization. Consequently, they only tangentially, if at all, promote other tissues within the skeletal system, including vascular and nerve tissues as well as inflammatory cells, all of which are key to bone remodeling. Indeed, bone healing involve complex and at times synergistic activities of multiple cell types including macrophages, neurons, mesenchymal stem cells (MSCs), and osteogenic cells. Interestingly, the data discussed above indicates that the 3D printed bioactive SiC orthopedic screw of the invention either through adhesion or Si ion release supported the activities of macrophages, neurons and osteoblasts, all cell types preeminently involved in skeletal tissue healing.

The silicate ions released from the SiC orthopedic screw appeared to have immunomodulatory effects as in their presence both macrophage proinflammatory cytokine secretions and ROS expression were inhibited. This contrasts with the effects of titanium ions released from Ti₆Al₄V scaffolds that were associated with increased macrophage TNFα secretions. Notably, elevated TNFα concentrations have been shown to inhibit osteoblast differentiation and activity through various mechanisms, including the downregulation of key osteogenic genes and the promotion of osteoblast apoptosis, leading to reduced bone formation. Moreover, it is demonstrated that bone cells cultured on porous bioactive SiC scaffold had higher osteogenic gene expression associated with an increased mineralization of extracellular matrix. The current demonstration of SiC coated with silica and/or silicate ions effects on macrophages, neurons and osteoblasts indicate their promotion of bone tissue formation at the interface with SiC implant.

Bone innervation is essential for regulating bone cell function and remodeling. Furthermore, the close interactions between blood vessels and nerve fibers play a pivotal role in enhancing development of bone with similar physicochemical and mechanical properties to that of the surrounding bone. Indeed, incorporating sensory nerve tracts into tissue-engineered bone grafts stimulated osteogenesis. Results of the present invention demonstrate both neuron growth and the generation of an extensive axon network through the entire porous surface of the 3D printed bioactive SiC screws without the addition of neuron activator factors. The observed enhanced axonal growth can be attributed to the surface chemistry and increased surface area of the 3D printed SiC orthopedic screws. Indeed, neurons cultured on a mesoporous substrate akin to SiC screws assessed here formed a greater number of axons compared to neurons cultured onto planar silicon substrates. Moreover, in skeletal tissue Mg ions released from bioceramics have been shown to directly promote neurogenesis along with osteogenesis through intercellular signaling mechanisms. Furthermore, doping borate glasses with iron, gallium, and zinc improved the outgrowth of neurons, whereas doping with iodine was detrimental to neurons. The data presented here highlight the need to account for neurogenic effects of ceramic ions in the regeneration of innervated bone.

Moreover, the data discussed above indicates that dissolved silicate ions at concentrations ranging from 25 to 150 ppm modulate the immune cells and stimulate innervation suggesting that the role of dissolved silicate ions go beyond the simple osteoconductivity, i.e., the ability to form hydroxyapatite surface layer that bonds to bone of silicate materials. In physiological conditions (i.e., pH˜7.3-7.4), and at concentrations below 2 mM (56 ppm Si), silicate exists predominantly as orthosilicic acid (Si(OH)₄). Si(OH)₄ is uncharged at neutral pH and tends to polymerize to form polysilicate at silicate concentration above 2-3 mM (56-84 ppm Si). The present invention is in line with the stimulatory effects of sodium silicate (up to 250 ppm) on mesenchymal stem cell proliferation and osteogenic differentiation. Moreover, dissolved Si was shown to promote cell membrane fluidity and enhance cell signaling, possibly through reactions between hydroxyl groups of the Si(OH)₄ and cell membrane fatty acid double bonds.

The present example demonstrates that surface modification of SiC enabled 3D printing of bioactive orthopedic screws. This method leads to new orthopedic screws with an enhanced hydroxyapatite layer on the material surface. The surface of the 3D printed SiC was bio-active and promoted neuron and osteoblast adhesion and activities including axonal growth. Moreover, Si ions released from 3D printed bioactive orthopedic screws inhibited macrophage proinflammatory activities and promoted both neuron and osteoblast adhesion and spread along with axon growth. These results suggest that the 3D printed bioactive SiC orthopedic screws of the invention interact with essential cells including macrophages, neurons and osteoblasts within the bone leading to an anti-inflammatory microenvironment and promoting both neurogenesis and osteogenesis critical to new bone formation.

Additionally, the 3D printed SiC device can be strengthened and densified by the spontaneous growth of silica nanowires inside the pores upon post processing heat treatment in the temperature range 500-1000° C. The nanowires can be functionalized with ammonia groups to capture carbon and carbon dioxide, with SEM image of the fracture surface of 3D printed SiC showing extensive growth of silica nanowires inside the pores following developed heat treatment protocol. Carbon capture will be enhanced by the huge surface area provided by the functionalized nanowires. Ammonia groups (NH₃) can be tethered on the huge surface are provided by the silica nanowires to capture carbon and carbon dioxide from air, green houses, and factories. See FIG. 17.

The SiC device can be used in lightweight mirrors and structural components for ground- and space-based imaging systems, such as telescopes and satellites. The ability to match materials between the mirrors and support structure is ideal because issues with mismatched coefficient of thermal expansion (CTE) and the corresponding thermal distortion caused by temperature variations and gradients are minimized, thus the present invention offers low CTE and high thermal conductivity required.

In addition, the SiC device of the present invention can be used in lithography positioning tools/stepper systems as the SiC device of the present invention provides the ability to integrate threaded inserts, mounting locations, and the wafer chuck with the stage mirrors with significant design freedom and enhanced performance. The SiC device can also be used in scanning mirror systems as the integration of lightweight, stiff mirrors with gimbal attachments and other structural features provides design freedom as well as assembly and alignment cost reductions.

The ability of the SiC device of the present invention to 3D print near-net shape geometries increases design freedom and reduces production cost and time for improved protection against ballistic threats. Applications include both vehicle and body armor (e.g., scan a soldier's geometry and use on-demand printing to produce customized plates). The SiC device of the present invention can also be used as heat shields and structural parts due to its ability to provide high stiffness, low density, low CTE, and high thermal conductivity.

Additionally, the SiC device of the present invention can be used in energy and nuclear power stations, as the 3D printed SiC device can be used in nuclear reactors due to its high thermal conductivity and resistance to radiation damage. Moreover, the 3D printed SiC device can offer customized designs for turbine blades and energy-efficient components such as solar panels and power electronics.

The SiC device of the present invention can be employed in automotive manufacturing for producing lightweight and durable components such as catalyst for exhaust filters engine parts, brake components, exhaust systems, and suspension parts, contributing to improved fuel efficiency and performance, as the SiC device provides high stiffness, low density, low CTE, and high thermal conductivity. Furthermore, the SiC device of the present invention can be employed in high-performance and long-range electrical vehicles as current conventional silicon-based power devices, such as Insulated Gate Bipolar Transistor, are not able to further reduce their heat dissipation, weight, and size, and these are all prerequisites for improvements in efficiency, which the SiC device of the present invention remedies. Furthermore, the imminent transition of high-voltage batteries from 400 V to 800 V imposes even more stringent voltage requirements on the power devices used. Additionally, the SiC device of the present invention can be employed in oil refinery, metal, and water purification.

The SiC device of the present invention provides biocompatibility and resistance to corrosion, which makes it suitable for medical applications such as implants, prosthetics, surgical instruments, and medical sensors. Further, the 3D printing allows for the fabrication of complex and customized medical devices.

The foregoing examples are illustrative of the present invention, and are not to be construed as limiting thereof. Although the invention has been described in detail with reference to preferred embodiments, variations and modifications exist within the scope and spirit of the invention as described and defined in the following claims.

1. A method of making a silicon carbide (SiC) device, comprising:
(i) preparing a SiC device;
(ii) thermally treating the SiC device at about 500-900° C.;
(iii) exposing the SiC device to NaOH to form a silica gel layer on the surface of the SiC device;
(iv) thermally treating the SiC device after exposing at about 550-1300° C.;
(v) exposing the SiC device to nitrogen to form silicon nitride on the SiC device surface; and
(vi) thermally treating the SiC device during or after exposing to nitrogen at about 900-1300° C.

2-5. (canceled)

6. The method of claim 1, wherein in step (i) the prepared device is composed only of SiC.

7. The method of claim 1, wherein in step (i) the prepared device is a material with a SiC coating.

8. (canceled)

9. The method of claim 1, wherein the SiC device is prepared by additive manufacturing (e.g., 3D printing), powder metallurgy, or chemical vapor deposition (CVD).

10-18. (canceled)

19. The method of claim 1, wherein in step (ii) the thermal treatment is carried out for about 2-8 hours.

20. (canceled)

21. The method of claim 1, wherein in step (ii) the thermal treatment is carried out in a carbon atmosphere or in the presence of graphite powder.

22. The method of claim 1, wherein exposing the SiC device to NaOH is carried out with about 15-20% NaOH.

23-25. (canceled)

26. The method of claim 1, wherein in step (iv) the thermal treatment carried out for about 2-8 hours.

27. (canceled)

28. The method of claim 1, wherein exposing the SiC device to nitrogen comprises exposing the SiC device to a nitrate compound, such as ammonium nitrate or sodium nitrate, e.g., at about room temperature, followed by thermal treatment.

29. The method of claim 1, wherein exposing the SiC device to nitrogen comprises exposing the SiC device to NH₄OH, e.g., at about room temperature, followed by thermal treatment.

30. The method of claim 1, wherein exposing the SiC device to nitrogen comprises exposing the SiC device to a nitrogen atmosphere, e.g., at about 900-1300° C.

31-32. (canceled)

33. The method of claim 1, wherein in step (vi) the thermal treatment is carried out for about 2-48 hours.

34. (canceled)

35. A method of increasing strength of a SiC device, comprising:
(i) exposing SiC particles to NaOH to create a silica gel layer;
(ii) drying the SiC particles in air to dry the silica-gel layer;
(iii) mixing the SiC particles with aluminium oxide and/or magnesium oxide and/or silicate mineral particles such as mullite, cordierite, cristobalite, or spodumene;
(iv) preparing a SiC device using the mixture of step (iii) using a 3D printer or by pressing in a mold; and
(v) thermally treating the SiC device at about 800-1400° C. for 4-24 hours.

36. The method of claim 35, wherein in step (i) exposing the SiC particles to NaOH is carried out with about 1-20% NaOH.

37. (canceled)

38. The method of claim 1, wherein the SiC device is a medical device.

39. The method of claim 38, wherein the SiC device is a screw, plate, stent, cage, rod, plug, pin, implant, mesh, or cable.

40. The method of claim 1, wherein the SiC device is a ground-based imaging system device or a space-based imaging system device; a lithography positioning tool or a stepper system device; a scanning mirror system device or optoelectronic device; a ballistic protection device; an energy or nuclear power device; a component of an automotive device such as catalyst, brake, exhaust, or suspension; a component in an electrical vehicle such as insulated gate bipolar transistor; or a component of a filter device.

41-47. (canceled)

48. A SiC device prepared by the method of claim 1.

49. A method of implanting or attaching a SiC device to a subject in need thereof, comprising using a SiC device prepared by the method of claim 1.

50. A method of stimulating innervation of tissue in a subject in need thereof, comprising contacting the tissue with a SiC device prepared by the method of claim 1.
